(19) United States
(12) Patent Application Publication
(10) Pub. No.: US 2026/0226250 A1
(43) Pub. Date: Aug. 6, 2026

(54) METHOD FOR CLEAVING URETHANES

(71) Applicant: Covestro Deutschland AG, Leverkusen (DE)

(72) Inventors: Martin Machat, Köln (DE); Jakob Marbach, Leverkusen (DE); Kai Laemmerhold, Odenthal (DE); Matthias Leven, Köln (DE); Wolf Kloeckner, Köln (DE); Simon Rengshausen, Leverkusen (DE); Sami El Sayed, Aachen (DE); Julian Schroers, Aachen (DE); Tugrul Nalbantoglu, Aachen (DE)

(21) Appl. No.: 19/154,925

(22) PCT Filed: Feb. 13, 2024
(86) PCT No.: PCT/EP2024/053609

§ 371 Date: Aug. 8, 2025

(30) Foreign Application Priority Data
Feb. 14, 2023 (EP) 23156544.1
Nov. 29, 2023 (EP) 23212855.3

(51) Int. Cl. C08J 11/24; C07C 269/06; C07C 269/08; C08J 11/16

(52) U.S. Cl. CPC C08J 11/24; C07C 269/06; C07C 269/08; C08J 11/16; C08J 2375/04

(57) ABSTRACT

The invention relates to a method for the chemical cleavage of urethanes, in particular polyurethanes, by reacting with a chemolysis alcohol (alcoholysis). The method is characterised in that a (poly)urethane is reacted with a chemolysis alcohol which has a boiling point of no greater than 200° C. at 1013 mbar, wherein a mass ratio of the chemolysis alcohol to the urethane is set between 0.50 and 15 and a chemolysis product containing a carbamate is obtained, wherein the chemolysis alcohol is selected from (i) an aliphatic secondary or tertiary (preferably secondary) monoalcohol, (ii) an aliphatic primary monoalcohol that is connected to two or three C atoms at (at least) one β position or γ-position (preferably β-position) other C atom relative to the hydroxy group of the monoalcohol, (iii) an aromatic monoalcohol or (iv) a mixture of two or more of the above-mentioned chemolysis alcohols.

The present invention relates to a process for chemically cleaving urethanes, especially polyurethanes, by reaction with a chemolysis alcohol. The process has the feature that a (poly)urethane is reacted with a chemolysis alcohol having a boiling point of not more than 200° C. at 1013 mbar, where a mass ratio of the chemolysis alcohol to the urethane of 0.50 to 15 is established and a chemolysis product comprising a carbamate is obtained, where the chemolysis alcohol is selected from (i) an aliphatic secondary or tertiary (preferably secondary) monoalcohol, (ii) an aliphatic primary monoalcohol bonded to two or three further carbon atoms at (at least) one carbon atom in the β position or γ position (preferably β position) to the hydroxy group of the monoalcohol, (iii) an aromatic monoalcohol or (iv) a mixture of two or more of the aforementioned chemolysis alcohols.

Urethanes are versatile products. Polyurethanes in particular enjoy a variety of applications in industry and in everyday life. In the case of the polyurethanes, distinctions are typically made between polyurethane foams and what are known as “CASE” products, with “CASE” being a collective term for polyurethane coatings (e.g., paints), adhesives, sealants and elastomers. The polyurethane foams are typically divided into rigid foams and flexible foams. Common to all these products in spite of their heterogeneity is the polyurethane base structure, which is formed by the polyaddition reaction of a polyfunctional isocyanate and a polyol and which, in the case, for example, of a polyurethane based on a diisocyanate O═C═N—R—N═C═O and a diol H—O—R′—O—H (where R and R′ denote organic residues), can be represented as

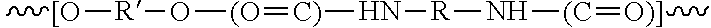

Even simple (nonpolymeric) urethanes feature the urethane bond. Polyurethanes have the greatest economic significance among the urethanes.

Many polyurethanes contain further structural units as well as the polyurethane base structure. These especially include urea, isocyanurate, allophanate and biuret structural units.

It is precisely the great economic success of the polyurethanes in particular that is responsible for the large amounts of polyurethane waste generated (for example from old mattresses, seating furniture or insulation materials) that must be sent to a sensible use. The mode of reuse that is the easiest to implement technically is that of incineration, with the heat of combustion released being utilized for other processes, examples being industrial processes. In this way, however, closure of the raw material cycles is impossible. Another mode of reuse is called “physical recycling”, which sees polyurethane wastes mechanically comminuted and used in the production of new products. There are naturally limits to this mode of recycling, and there has therefore been no lack of attempts to recover the underlying raw materials of polyurethane production by redissociation of the urethane bonds (and of any binding structures additionally present, such as isocyanurate, urea, allophanate biurets bonds) (called “chemical recycling”). The raw materials to be recovered primarily comprise polyols (i.e., in the above example, H—O—R′—O—H) or degradation products thereof (for example the parent monomers of a polyester polyol). In addition, it is also possible through hydrolytic cleavage of the urethane bond to recover amines (i.e., in the example above, H₂N—R—NH₂) which can be phosgenated to afford isocyanates (in the aforementioned example to afford O═C═N—R—N═C═O) after workup.

A variety of chemical recycling approaches have been developed in the past. The three most important are briefly summarized as follows:
1. Hydrolysis of urethanes by reaction with water to recover amines and polyols with formation of carbon dioxide.
2. Glycolysis of urethanes by reaction with alcohols, with replacement of the polyols incorporated in the urethane groups by the alcohol used and hence release of the polyols. This process is commonly referred to in the literature as transesterification (more accurately: transurethanization). Regardless of the exact nature of the alcohol used, this mode of chemical recycling is dubbed glycolysis in the literature, a term that really applies only for glycol or glycol derivatives. (In the context of the present invention, the term alcoholysis is therefore generally used.) A glycolysis may be followed by a hydrolysis. If the hydrolysis is conducted with the direct process product from the glycolysis (i.e. without prior separation of polyols and carbamates), this is referred to as
3. Hydroglycolysis (hydroalcoholysis) of urethane bonds by reaction with alcohols and water. It is of course likewise possible to add alcohol and water from the start, in which case the above-described processes of glycolysis and hydrolysis proceed in parallel.

A summary of the known methods of polyurethane recycling is offered by the review article by Simón, Borreguero, Lucas and Rodriguez in Waste Management 2018, 76, 147-171 [1].

WO 2022/171586 A1 describes a process for recovering raw materials (i.e. polyols and optionally also amines) from polyurethane foams comprising a chemolysis. The chemolysis has the feature that a polyurethane foam is reacted with an alcohol and water in the presence of a catalyst at a temperature in the range from 130° C. to 195° C., wherein the mass ratio of (altogether employed) alcohol and (altogether employed) water on the one hand to the polyurethane foam on the other hand is in the range from 0.5 to 2.5 and wherein the mass of the water is 4.0% to 10% of the mass of the alcohol. The catalyst comprises a metal salt selected from a carbonate, a hydrogencarbonate, an orthophosphate, a monohydrogenorthophosphate, a metaphosphate or a mixture of two or more of the aforementioned metal salts.

WO 2023/285545 A1 describes a process for recycling a polyurethane, in which the polyurethane is reacted with a first alcohol and the low molecular weight carbamate formed therein or in a further transurethanization with a second alcohol is cleaved thermally to the parent alcohol and the parent isocyanate. Particularly preferred first and/or second alcohols are propanol, pentanol, isopropyl alcohol, butanol, hexanol, nonanol, octanol, glycerol, ethylene glycol, diethylene glycol and triethylene glycol. In the examples, a flexible polyurethane foam is reacted once with octanol (mass ratio of foam to alcohol 1:2, under reflux in an apparatus operated at ambient pressure) and once with iso-propanol (mass ratio of foam to alcohol 1:5, in a pressure reactor at an argon pressure of bar).

EP 3 590 999 B1 describes a process for degrading plastics with methanol or ethanol in the presence of methoxide as catalyst. The examples describe the degradation of flexible polyurethane foam.

U.S. Pat. No. 4,316,992 describes a process for recovering polyether polyols from polyurethane foams. In the process, the polyurethane foam is dissolved in an alcohol at 225° C. to 280° C., then superheated steam is passed through the solution thus obtained at 185° C. to 220° C. Suitable alcohols mentioned are saturated mono- or polyalcohols having boiling points between 225° C. and 280° C. These may be straight-chain, branched, cyclic or aromatic. Diols or triols having ether bridges are preferred, especially diethylene glycol, dipropylene glycol, dibutylene glycol, glycerol and propylene ethylene glycol, of which diethylene glycol is emphasized as being particularly advantageous. All that is shown in practical terms is the use of diethylene glycol (examples 1 and 2), glycerol (example 3) and hexane-1,6-diol (example 4). No correlation between the choice of alcohol and the formation of by-products can be inferred from the patent specification.

Patent specification U.S. Pat. No. 4,336,406 describes hydroalcoholysis of polyurethane foams by dissolving in an alcohol at 225° C. to 280° C., followed by reaction with water (liquid water here) at 185° C. to 220° C. Suitable alcohols described are saturated alcohols having a boiling point between 225° C. and 280° C.; diethylene glycol is preferred. In this case too, no correlation between the choice of alcohol and the formation of by-products can be inferred from the patent specification.

U.S. Pat. No. 5,104,932 describes a process for recovering improved asphalt or bitumen compositions comprising polymeric residues. What is disclosed for this purpose is a partial alcoholysis (for example with tert-butyl alcohol), hydrolysis or hydroalcoholysis of polyurethane and/or polyester wastes, followed by reaction of the resultant polymer residue with molten asphalt and/or bitumen.

US 2016/0145409 A1 describes a process for recovering organic fibers from composite materials comprising a polymer matrix and organic fibers. The process comprises conversion of the composite material in an alcohol-water mixture. The polymer is especially an epoxy resin or phenolic resin. Water and alcohol are preferably used here in equal volumes, and the alcohol is preferably selected from methanol, ethanol, n-propanol, iso-propanol, glycerol or a mixture of these.

DE 42 17 024 A1 describes a process for recovering polyols from polyurethane plastics, polyurethaneurea plastics and/or polyurea plastics, in which the plastics are reacted with a di- and/or polyfunctional alcohol and water. The only alcohols described are glycols.

EP 0 601 596 A1 describes a process for recovering polyols from polyurethane foams by glycolysis. It is a feature of the process that primary amines that are formed in the glycolysis owing to the presence of urea bonds in the polyurethane foam are converted to secondary amines by an alkoxylation reaction.

WO 2010/130652 A2 describes a process for hydrolyzing isocyanate adducts, which is understood in the terminology of this published specification to mean in particular residues from isocyanate production (e.g. distillation residue from the production of tolylene diisocyanate). The hydrolysis is performed in the presence of an imidazole. If the isocyanate adduct is a residue from isocyanate synthesis (=not a polyurethane), hydrolysis may be preceded by contacting with a primary or secondary alcohol.

The article “tert-Amyl Alcohol-Mediated Deconstruction of Polyurethane for Polyol and Aniline Recovery” by Martin B. Johansen et al. in ACS Sustainable Chem. Eng., 2022, 10 (34), 11191-11202 describes the chemolysis of polyurethanes with tert-amyl alcohol to directly form the amine that was the precursor of the isocyanate used in the polyurethane synthesis. What is proposed is a reaction mechanism in which the polyurethane used is first cleaved back thermally to isocyanate and polyol, and isocyanate thus formed forms a carbamate as intermediate with the amyl alcohol. The carbamate reacts with elimination of 2-methyl-1-butene and 2-methyl-2-butene to form unstable carbamic acid, from which the amine is formed with release of carbon dioxide. The reactions are effected with a mass ratio of alcohol to polyurethane of 16 at temperatures of 200 to 225° C. There is no discussion of any influence of the choice of chemolysis alcohol on the formation of N-alkylated by-products.

Only a few of the chemical recycling processes known from the literature are in sustained operation on an industrial scale; many have not even reached pilot scale [1]. In view of generally increased environmental awareness and increased efforts to configure industrial processes to be as sustainable as possible-both of which are fundamentally in favor of chemical recycling—this shows clearly that the chemical recycling of polyurethane products is still by no means mature from a technical and economic point of view. Challenges exist particularly with regard to the purity of the products recovered. Polyols must be recovered without amine impurities if at all possible, in order, for instance, not to adversely affect foaming characteristics in the case of reuse in the production of polyurethane foams. If another aim is recovery of amines, these must of course also be obtained in maximum purity. In addition, the polyurethane products to be reutilized usually still contain various auxiliaries and additives (stabilizers, catalysts, flame retardants and the like), which have to be separated from the actual target products of the recycling and disposed of in an economically viable and environmentally benign manner. Moreover, an economic recycling process must ensure that the reagents used (for example alcohols used) can be recovered and reused (i.e. follow a closed loop) as completely as possible. Due to the large volumes of polyurethane waste generated from used polyurethane foams (for example mattresses, seating furniture, vehicle seats, insulation materials and the like), the recycling of polyurethane foams is of particular importance.

A further important aspect relates to the avoidance of by-products during chemolysis. Troublesome by-products may especially be N-alkylated compounds. Such compounds may form, for example, in the industrial production process of di- and polyamines of the diphenylmethane series, the aminic precursors of the di- and polyisocyanates of the diphenylmethane series. It is known here that an elevated concentration of N-alkylated products leads to an elevated HC value of the isocyanates produced. Fixed HC limits are applicable depending on the field of use of the isocyanates. The result of this is that even substrate streams wherein the proportion of N-alkylated groups relative to the free aminic groups is not significantly below 1% are generally unsuitable for a conventional phosgenation reaction since the product streams will no longer meet the necessary specifications. The recovery of aromatic amines from polymeric waste streams in the form of chemical recycling enables additional reaction pathways for formation of N-alkylated compounds. The resulting by-products will have a correspondingly adverse effect on the phosgenation process. This aspect has not been sufficiently taken into account to date in the prior art.

There was therefore a need for further improvements in the field of chemolysis of urethanes, especially of polyurethanes. In particular, it would be desirable to as far as possible suppress the formation of N-alkylated compounds in the chemolysis.

Taking account of this requirement, the present invention provides a process for chemolyzing (=chemically cleaving) a urethane (especially polyurethane) based on an isocyanate component and an alcohol component by reaction with a chemolysis alcohol to form a carbamate of an isocyanate of the isocyanate component and of the chemolysis alcohol,
especially for the purpose of recovering starting materials for the production of chemical products,
said process comprising the steps of:
(A) providing the urethane and
(B) chemolyzing the urethane from (A) with the chemolysis alcohol in the presence or absence of a chemolysis catalyst, where the chemolysis alcohol at 1013 mbar has a boiling point of not more than 200° C. (preferably from 82° C. to 200° C., more preferably from 82° C. to 185° C.), and where a mass ratio of the chemolysis alcohol to the urethane, m(chemolysis alcohol)/m(urethane) (with m=mass), in the range from 0.50 to 15 is established (and, consequently, the chemolysis alcohol is in stoichiometric excess in relation to the urethane groups), to form a chemolysis product comprising the carbamate,
   wherein the chemolysis alcohol is selected from (i) an aliphatic secondary or tertiary (preferably secondary) monoalcohol, (ii) an aliphatic primary monoalcohol bonded to two or three further carbon atoms at (at least) one carbon atom in the β position or γ position (preferably β position) to the hydroxy group of the monoalcohol, (iii) an aromatic monoalcohol or (iv) a mixture of two or more of the aforementioned chemolysis alcohols.

It has been found in an entirely surprising manner that the use of branched monoalcohols as described above as chemolysis reagents can suppress or even entirely prevent the formation of N-alkylated by-products. The invention therefore further provides for the use of a monoalcohol having a boiling point at 1013 mbar of not more than 200° C. (preferably from 82° C. to 200° C., more preferably from 82° C. to 185° C.) and selected from
(i) an aliphatic secondary or tertiary (preferably secondary) monoalcohol, (ii) an aliphatic primary monoalcohol bonded to two or three further carbon atoms at (at least) one carbon atom in the β position or γ position (preferably β position) to the hydroxy group of the monoalcohol, (iii) an aromatic monoalcohol or (iv) a mixture of two or more of the aforementioned monoalcohols
   as chemolysis alcohol in a chemolysis (chemical cleavage) of a urethane (especially polyurethane) based on an isocyanate component and an alcohol component in a mass ratio of the chemolysis alcohol to the urethane, m(chemolysis alcohol)/m(urethane), in the range from 0.50 to 15, preferably 0.75 to 10, more preferably 1.0 to 5.0 (and, consequently, the chemolysis alcohol is in stoichiometric excess in relation to the urethane groups), to form a carbamate of an isocyanate of the isocyanate component and the chemolysis alcohol,
   to reduce the formation of N-alkylated by-products, i.e. of by-products in which (at least) one organic residue is bonded to a nitrogen atom via an aliphatic carbon atom (especially originating from the chemolysis alcohol or the alcohol component).

All aspects described in connection with the process of the invention (for example preferred chemolysis alcohols, temperatures, pressures, ratios of the feedstocks etc.) are of course also applicable to the use of the invention, and there will be no further separate reference thereto for avoidance of superfluous repetition.

According to the invention, the mass ratio of the chemolysis alcohol to the urethane is at least 0.50:1 and at most 15:1, preferably at least 0.75:1 and at most 10:1, more preferably 1.0:1 and at most 5.0:1.

In the terminology of the present invention, the term isocyanates encompasses all the isocyanates known in the specialist field in connection with urethane chemistry. The expression “an isocyanate” does of course also encompass embodiments in which two or more different isocyanates (e.g. mixtures of MDI and TDI) were used in the production of the (poly)urethane, unless explicitly stated otherwise, for instance by the wording “exactly one isocyanate”. The entirety of all isocyanates used in the production of the (poly)urethane is referred to as the isocyanate component (of the (poly)urethane). The isocyanate component comprises at least one isocyanate. Analogously, the entirety of all mono- or polyols used in the production of the (poly)urethane is referred to as the alcohol component (of the (poly)urethane). The alcohol component comprises at least one mono- or polyol.

In the terminology of the present invention, the term mono- or polyols encompasses all mono- or polyols known in the specialist field in connection with urethane chemistry. The expression “a monool” or “a polyol” does of course also encompass embodiments in which two or more different mono- or polyols were used in the production of the urethane. Therefore, if reference is made hereinafter, for example, to “a polyether polyol” (or “a polyester polyol” etc.), this terminology does of course also encompass embodiments in which two or more different polyether polyols (or two or more different polyester polyols etc.) were used in the production of the (poly)urethane.

In the terminology of the present invention, the carbon atom bearing the hydroxy group of the monoalcohol is referred to as the α carbon atom. The next carbon atom is in the 8 position to the hydroxy group, the next carbon atom but one in the γ position etc.

The chemolysis of the urethane from (A) with the chemolysis alcohol is performed in a mass ratio of the chemolysis alcohol to the urethane, m(chemolysis alcohol)/m(urethane), in the range from 0.50 to 15. The mass ratio m(chemolysis alcohol)/m(urethane) may assume any value in this range including the endpoints. It is also possible to employ a smaller selection range within the range from 0.50 to 15, for instance 0.75 to 15 or 0.75 to 10 or 1.0 to 10 or 1.0 to 5.0 or 1.0 to 4.8 or 1.0 to 4.5 or 1.0 to 4.4. It is also possible to employ selection ranges that result from combination of aforementioned upper and lower limits, for instance 0.50 to 4.8 or 0.50 to 4.5 or 0.50 to 4.4 or 0.75 to 4.8 or 0.75 to 4.5 or 0.75 to 4.4. Any selection range of the mass ratio m(chemolysis alcohol)/m(urethane) may be combined with any embodiment/possible configuration/variant of the invention.

In the terminology of the present invention, carbamates refer to the urethanes formed on performance of the chemolysis as a result of the reaction with the chemolysis alcohol, in order to be able to distinguish them from the urethane used. This choice of terminology serves merely to simplify the discussion.

An amine corresponding to an isocyanate is the amine that can be phosgenated to obtain the isocyanate according to R—NH₂+COCl₂→R—N═C═O+2 HCl.

In a further embodiment of the embodiments described below, the chemolysis of the invention is performed in the absence of a chemolysis catalyst. What this means in the context of the present invention is that only the chemolysis alcohol, but no chemolysis catalyst, is added to the (poly)urethane to be cleaved. It is known that polyurethane foams, for example, may still contain residual constituents of foaming catalysts (for example tertiary amines). The presence of such catalysts originating from the original use of the (poly)urethane to be cleaved does not constitute a departure from the scope of this embodiment of the present invention. All that is essential in this connection is that no additional catalysts (more specifically: no compounds that would catalyze chemolysis, i.e. no chemolysis catalysts) are added via the catalysts that are present anyway in some cases from the original use of the (poly)urethanes to be cleaved. The present examples were conducted with a model urethane that was free of foaming catalysts such as those specified above.

There will firstly follow a brief summary of various possible embodiments of the invention:

In a first embodiment of the invention, which is combinable with all other embodiments apart from those that do not envisage isolation of the carbamate, the process comprises step (C):
(C) separation of the carbamate formed in (B) from the chemolysis product using an extraction with an organic solvent, optionally with addition of water, and/or a solid-liquid phase separation to obtain a liquid alcohol phase (especially a polyol phase) as well as the carbamate.

In a second embodiment of the invention, which is a particular configuration of the first embodiment, the carbamate separated off in (C), optionally after purification, is converted further in a step (D) to obtain a chemical product, where (D) comprises one of the following reactions:
(D.I) hydrolysis of the carbamate in the presence or absence of a hydrolysis catalyst to form an amine corresponding to the isocyanate of the isocyanate component;
(D.II) hydrogenolysis of the carbamate in the presence of a hydrogenolysis catalyst to form an amine corresponding to the isocyanate of the isocyanate component;
(D.III) cleaving of the carbamate in the presence or absence of a carbamate cleavage catalyst to the isocyanate of the isocyanate component and the chemolysis alcohol; or
(D.IV) reaction of the carbamate with a polyol in the presence or absence of a catalyst to give a further OH-terminated carbamate, especially—if the urethane from step (A) is a polyurethane—to give an OH-terminated prepolymer.

In a third embodiment of the invention, which is combinable with all other embodiments apart from those that envisage isolation of the carbamate, the process additionally comprises the steps of:
(B.I) hydrolyzing the carbamate formed in (B) in the presence or absence of a hydrolysis catalyst to form an amine by reaction of the (unchanged) chemolysis product obtained in (B) with water to obtain a hydrolyzed product mixture; and
(C.I) extracting the hydrolyzed product mixture with an organic (especially halogenated) solvent to obtain an amine phase and a liquid alcohol phase (especially a polyol phase).

In a fourth embodiment of the invention, which is combinable with all embodiments that envisage hydrolysis of the carbamate, the hydrolysis is performed in the presence of a hydrolysis catalyst comprising
(I) an (organic or inorganic) Brønsted base selected from (i) a hydroxide (especially sodium hydroxide, tetramethylammonium hydroxide, potassium hydroxide or tetrabutylammonium hydroxide), (ii) a carbonate (especially an alkali metal carbonate, for example sodium or potassium carbonate), (iii) a hydrogencarbonate (especially an alkali metal hydrogencarbonate such as sodium or potassium hydrogencarbonate), (iv) an orthophosphate or metaphosphate, preferably orthophosphate (especially alkali metal phosphate or alkali metal hydrogenphosphate) or (v) a mixture of two or more of the aforementioned Brønsted bases.
   and/or
(II) a urethanase, especially one of the urethanases described in EP 3 587 570 A1.

In a fifth embodiment of the invention, which is a particular configuration of the second embodiment, step (D.II) is performed, where the hydrogenolysis catalyst comprises copper, palladium (especially Pd/C, PdCl₂ or Pd(OAc)₂), nickel (especially Raney nickel), manganese (especially Mn complexes having a tridentate chelate ligand that binds via P and N donor atoms, and CO and/or halogen ligands) or platinum (especially platinum (IV) oxide).

In a sixth embodiment of the invention, which is a further particular configuration of the second embodiment, step (D.III) is performed, wherein the cleavage of the carbamate is performed in the presence of a carbamate cleavage catalyst comprising
(I) a metal-free or metal-containing Brønsted- or Lewis-acidic catalyst
   or
(II) a metal-free or metal-containing Brønsted- or Lewis-basic catalyst.

In a seventh embodiment of the invention, which is a further particular configuration of the second embodiment, step (D.IV) is performed, wherein the reaction of the carbamate with the polyol is performed in the presence of a catalyst comprising
a carbonate, a hydrogencarbonate, a hydroxide, an orthophosphate, a monohydrogenorthophosphate, a metaphosphate, an orthovanadate (where all the aforementioned chemolysis catalysts are preferably used in the form of their sodium or potassium salts), a titanium alkoxide (especially tetra-n-butyl titanate, Ti(O-nBu)₄), a tertiary amine (especially 1,4-diazabicyclo(2.2.2) octane, “DABCO”), cesium fluoride, a stannate (especially dibutyltin dilaurate, “DBTL”, or monobutyltin oxide, n-BuSn(O)OH, “MBTO”) or a mixture of two or more of the aforementioned chemolysis catalysts.

In an eighth embodiment of the invention, which can be combined with all embodiments comprising step (C) or (C.I), the liquid alcohol phase from (C) or (C.I), in a step (E), is distilled and/or stripped to obtain (at least) one chemical product selected from (i) an alcohol of the alcohol component and/or (ii) a reaction product formed from an alcohol of the alcohol component in the chemolysis (B).

In a ninth embodiment of the invention, which can be combined with all embodiments, provided that they do not envisage catalytic performance of step (B), the chemolysis of the urethane (especially polyurethane) is performed in the absence of a chemolysis catalyst.

In a tenth embodiment of the invention, which can be combined with all embodiments, provided that they do not envisage uncatalyzed performance of step (B), the chemolysis of the urethane (especially polyurethane) is performed in the presence of a chemolysis catalyst, where the chemolysis catalyst comprises a carbonate, a hydrogencarbonate, a hydroxide, an orthophosphate, a monohydrogenorthophosphate, a metaphosphate, an orthovanadate (where all the aforementioned chemolysis catalysts are preferably used in the form of their sodium or potassium salts), a titanium alkoxide (especially tetra-n-butyl titanate, Ti(O-nBu)₄), a tertiary amine (especially 1,4-diazabicyclo(2.2.2) octane, “DABCO”), cesium fluoride, a stannate (especially dibutyltin dilaurate, “DBTL”, or monobutyltin oxide, n-Bu—Sn(O)OH, “MBTO”) or a mixture of two or more of the aforementioned chemolysis catalysts.

In an eleventh embodiment of the invention, which can be combined with all embodiments except for those that rule out the use of an aromatic chemolysis alcohol, the chemolysis alcohol is selected from iso-propanol, sec-butanol, iso-butanol, tert-butanol, 2-pentanol, 3-pentanol, iso-amyl alcohol (3-methyl-1-butanol), 2-methyl-2-butanol, neo-pentanol (2,2-dimethyl-1-propanol), 2-methyl-1-butanol, 3-methyl-2-butanol, 2-methyl-1-butanol, cyclopentanol, hexan-2-ol, hexan-3-ol, 2-methylpentan-1-ol, 2-methylpentan-2-ol, 2-methylpentan-3-ol, 4-methylpentan-2-ol, 3-methylpentan-1-ol, 3-methylpentan-2-ol, 3-methylpentan-3-ol, 2,2-dimethylbutan-1-ol, 3,3-dimethylbutan-1-ol, 3,3-dimethylbutan-2-ol, 2,3-dimethylbutan-1-ol, 2,3-dimethylbutan-2-ol, 2-ethylbutan-1-ol, 4-methylpentan-2-ol, cyclohexanol, phenol, 2-ethylhexan-1-ol or a mixture of two or more of the aforementioned chemolysis alcohols. (The following are preferred: iso-propanol, sec-butanol, iso-butanol, 2-pentanol, 3-pentanol, iso-amyl alcohol (3-methyl-1-butanol), neo-pentanol (2,2-dimethyl-1-propanol), 2-methyl-1-butanol, 3-methyl-2-butanol, pentaerythritol, 2-methyl-1-butanol, cyclopentanol, hexan-2-ol, hexan-3-ol, 2-methylpentan-1-ol, 2-methylpentan-3-ol, 4-methylpentan-2-ol, 3-methylpentan-1-ol, 3-methylpentan-2-ol, 2,2-dimethylbutan-1-ol, 3,3-dimethylbutan-1-ol, 3,3-dimethylbutan-2-ol, 2,3-dimethylbutan-1-ol, 2-ethylbutan-1-ol, 4-methylpentan-2-ol, cyclohexanol, phenol and 2-ethylhexanol.)

In a twelfth embodiment of the process of the invention, which can be combined with all embodiments except for those that envisage the use of an aromatic chemolysis alcohol, the chemolysis alcohol is selected from (i) an aliphatic secondary or tertiary (preferably secondary) monoalcohol, (ii) an aliphatic primary monoalcohol bonded to two or three further carbon atoms at (at least) one carbon atom in the β position or γ position (preferably β position) to the hydroxy group of the monoalcohol, or (iv) a mixture of two or more of the aforementioned chemolysis alcohols.

In a thirteenth embodiment of the invention, which is a particular configuration of the twelfth embodiment, the chemolysis alcohol is selected from sec-butanol, iso-butanol, iso-amyl alcohol (3-methyl-1-butanol), neo-pentanol (2,2-dimethyl-1-propanol), 2-methyl-1-butanol, cyclopentanol, 4-methylpentan-2-ol, cyclohexanol and 2-ethylhexanol. (The following are preferred: iso-propanol, 2-butanol, neo-pentanol (2,2-dimethyl-1-propanol), 2-methyl-1-butanol, iso-amyl alcohol (3-methyl-1-butanol), 2-ethyl-1-butanol and cyclohexanol. The following are particularly preferred: iso-propanol, 2-butanol, neo-pentanol (2,2-dimethyl-1-propanol), 2-methyl-1-butanol, 2-ethyl-1-butanol and cyclohexanol.)

In a fourteenth embodiment of the invention, which can be combined with all embodiments, the isocyanate component comprises an isocyanate selected from phenyl isocyanate (PHI; producible by phosgenation of aniline, ANL), tolylene diisocyanate (TDI; producible by phosgenation of tolylenediamine, TDA), the di- and polyisocyanates of the diphenylmethane series (MDI; producible by phosgenation of the di- and polyamines of the diphenylmethane series, MDA), pentane 1,5-diisocyanate (PDI; producible by phosgenation of pentane-1,5-diamine, PDA), hexamethylene 1,6-diisocyanate (HDI; producible by phosgenation of hexamethylene-1,6-diamine, HDA), isophorone diisocyanate (IPDI; producible by phosgenation of isophoronediamine, IPDA), diisocyanatodicyclohexylmethane (“saturated methylene diphenyl diisocyanate”, H₁₂MDI, especially the 4,4′isomer; producible by phosgenation of diaminodicyclohexylmethane, H₁₂MDA, obtainable in turn by ring hydrogenation of bicyclic MDA), xylylene diisocyanate (XDI; producible by phosgenation of xylylenediamine, XDA), para-phenylene diisocyanate (PPDI; producible by phosgenation of para-phenylenediamine) or a mixture of two or more of the aforementioned isocyanates. (The isocyanate component preferably comprises tolylene diisocyanate, di- and polyisocyanates of the diphenylmethane series, or a mixture of tolylene isocyanate and the di- and polyisocyanates of the diphenylmethane series, and especially does not include any other isocyanates aside from the aforementioned.)

In a fifteenth embodiment of the invention, which can be combined with all embodiments, the alcohol component comprises a mono- and/or polyol selected from
a polyether monool, a polyether polyol, a polyester polyol, a polyetherester polyol, a polyacrylate polyol, a polycarbonate polyol, a polyethercarbonate polyol or a mixture of two or more of the aforementioned polyols. (The alcohol component preferably comprises a polyester polyol, polyether polyol and/or a polyetherester polyol, more preferably a polyether polyol. Most preferably, the alcohol component is a polyether polyol (i.e. does not contain any mono- or polyols other than polyether polyols; but a mixture of two or more different polyether polyols is encompassed and does not depart from the scope of this embodiment).)

In a sixteenth embodiment of the invention, which can be combined with all embodiments, the urethane is a polyurethane.

In a seventeenth embodiment of the invention, which can be combined with all embodiments, step (B) is performed within a temperature range from 150° C. to 270° C., preferably 170° C. to 250° C., more preferably 180° C. to 230° C., and at a pressure in the range from 1.0 bar (especially ambient pressure) to 85 bar (where pressure and temperature are especially matched to one another such that the chemolysis can be implemented under reflux of the chemolysis alcohol chosen).

In an eighteenth embodiment of the invention, which can be combined with all embodiments, the mass ratio of the chemolysis alcohol to the urethane is chosen so as to give a molar ratio n (chemolysis alcohol)/n (urethane groups) (with n=molar amount) of 4.5 to 30, preferably 5.0 to 20, more preferably 8.5 to 15. If required, the molar amount of urethane groups, n (urethane groups), is determined by hydrolyzing a sample of the urethane provided in (A) and then determining the amine value by titration with 0.1 molar perchloric acid.

In a nineteenth embodiment of the invention, which can be combined with all embodiments, the chemolysis in step (B) is performed in a reactor which is inertized prior to commencement of the chemolysis with an inert gas, especially nitrogen, preferably with establishment of a partial inert gas pressure of 1.0 bar (especially ambient pressure) to 20 bar, preferably to 10 bar.

In a twentieth embodiment of the invention, which can be combined with all embodiments, in (B), a mass ratio of the chemolysis alcohol to the urethane, m(chemolysis alcohol)/m(urethane) (with m=mass), in the range from 0.50 to 4.5, especially 0.50 to 4.0, or in the range from 1.0 to 4.5, especially 1.0 to 4.0, is established.

The embodiments briefly outlined above and further possible configurations of the invention are elucidated in detail hereinafter. All the above-described embodiments and the further configurations of the invention described below are mutually and collectively combinable as desired unless the opposite is clearly apparent from the context to a person skilled in the art or unless expressly stated otherwise.

### Provision of the (Poly) Urethane for Chemical Recycling

Step (A) comprises providing the (poly)urethane to be chemically recycled in preparation for the chemolysis. This may in principle be any kind of urethane.

Preference is given to polyurethanes, i.e. urethanes that derive from polyisocyanates (2 or more isocyanate groups per molecule) and polyols (two or more alcohol groups per molecule). This may in principle be any kind of polyurethane, i.e. either polyurethane foams or polyurethane products from the CASE applications described at the outset. The polyurethane foams may be either flexible foams or rigid foams, preference being given to flexible foams (for example from used mattresses, furniture cushioning or car seats). Polyurethane foams are typically produced using blowing gases such as pentane or carbon dioxide. In the case of polyurethanes from CASE applications, preference is given to polyurethane elastomers, polyurethane adhesives and polyurethane coatings.

Preference is given to those urethanes or polyurethanes wherein the isocyanate component comprises an isocyanate selected from
phenyl isocyanate (PHI; producible by phosgenation of aniline, ANL), tolylene diisocyanate (TDI; producible by phosgenation of tolylenediamine, TDA), the di- and polyisocyanates of the diphenylmethane series (MDI; producible by phosgenation of the di- and polyamines of the diphenylmethane series, MDA), pentane 1,5-diisocyanate (PDI; producible by phosgenation of pentane-1,5-diamine, PDA), hexamethylene 1,6-diisocyanate (HDI; producible by phosgenation of hexamethylene-1,6-diamine, HDA), isophorone diisocyanate (IPDI; producible by phosgenation of isophoronediamine, IPDA), diisocyanatodicyclohexylmethane (“saturated methylene diphenyl diisocyanate”, H₁₂MDI, especially the isomer; producible by phosgenation of 4,4′diaminodicyclohexylmethane, H₁₂MDA, obtainable in turn by ring hydrogenation of bicyclic MDA), xylylene diisocyanate (XDI; producible by phosgenation of xylylenediamine, XDA), para-phenylene diisocyanate (PPDI; producible by phosgenation of para-phenylenediamine, PPDA) or a mixture of two or more of the aforementioned isocyanates.

In the case of the polyurethanes, it is particularly preferable that the isocyanate component comprises tolylene diisocyanate, di- and polyisocyanates of the diphenylmethane series or a mixture of tolylene diisocyanate and the di- and polyisocyanates of the diphenylmethane series, and especially does not comprise any further isocyanates aside from the above.

With regard to the alcohol component, this preferably comprises a mono- and/or polyol selected from
a polyether monool, a polyether polyol, a polyester polyol, a polyetherester polyol, a polyacrylate polyol, a polycarbonate polyol, a polyethercarbonate polyol or a mixture of two or more of the aforementioned polyols.

The alcohol component preferably comprises a polyester polyol, polyether polyol and/or a polyetherester polyol, more preferably a polyether polyol. Most preferably, the alcohol component is a polyether polyol (i.e. does not contain any mono- or polyols other than polyether polyols; but a mixture of two or more different polyether polyols is encompassed and does not depart from the scope of this embodiment).

A polyether polyol may also be one that is filled with a styrene-acrylonitrile copolymer (SAN copolymer).

Preferably, even step (A) comprises preparatory steps for the cleavage of the urethane bonds in step (B). In the case of polyurethanes, these are especially mechanical comminution. Such preparatory steps are known to a person skilled in the art; reference is made by way of example to the literature cited in [1]. Depending on the characteristics of the polyurethane (especially in the case of polyurethane foams), it can be advantageous to “freeze” this before the mechanical comminution in order to facilitate the comminuting operation.

It is also conceivable to conduct the above-described preparatory steps at a site spatially separate from the site of the chemolysis. In that case, the prepared foam is transferred into suitable transport vehicles, for example silo vehicles, for further transport. For further transport the prepared foam may additionally be compressed to achieve a higher mass-to-volume ratio. The foam is then transferred into the reaction apparatus provided for the chemolysis at the site of the chemolysis. It is also conceivable to connect the transport vehicle used directly to the reaction apparatus.

### Chemolysis of the (Poly)urethane

The chemolysis of the (poly)urethane, step (B), is preferably performed with exclusion of oxygen. This means that the reaction is carried out in an inert gas atmosphere (especially in a nitrogen, argon or helium atmosphere). It is also preferable that the chemolysis alcohol used is freed of oxygen by inert gas saturation.

The chemolysis is performed at reaction temperatures of 150° C. to 270° C., preferably 170° C. to 250° C., more preferably 180° C. to 230° C. The chemolysis is preferably performed in a pressure-rated reactor (autoclave) without pressure equalization, especially in such a way that the reaction is implemented “under reflux” (chemolysis alcohol evaporated, condenses in cooler regions of the reactor and flows back into the reacting mixture). In this procedure, a pressure which is established in the gas phase above the liquid reaction mixture corresponds to the vapor pressure of the chemolysis alcohol used at the prevailing temperature, i.e., depending on the nature of the chemolysis alcohol and the temperature, the pressure is in the range from about 1.0 bar to 85 bar (absolute). It is alternatively possible to conduct the chemolysis with pressure equalization, i.e. at ambient pressure. It is preferable to inertize the reactor used for the chemolysis prior to commencement of the chemolysis with an inert gas, especially nitrogen. The inert gas preferably establishes a partial inert gas pressure of 1.0 bar (especially ambient pressure) to 20 bar, preferably to bar (absolute).

An example of a suitable reactor is a stirred tank reactor, which may also be operated continuously. The use of a cascade of multiple continuously operated stirred tank reactors, optionally with a downstream tubular reactor (especially operated with plug flow), is likewise possible.

According to the invention, the mass ratio of the chemolysis alcohol to the (poly)urethane is in the range from 0.50 to 15 (it is, for example, in the range from 0.50 to 4.5 or 1.0 to 4.5 or 0.50 to 4.0 or 1.0 to 4.0). It is thus possible to limit the mass of chemolysis alcohol to be used, which is highly advantageous from a process engineering point of view (lower costs, lower complexity of workup). With regard to the molar ratios, these are regularly superstoichiometric with the mass ratios specified, such that complete conversion can be assured. The mass ratios are preferably chosen within the ranges mentioned so as to ensure a molar excess of the chemolysis alcohol (i.e. a molar ratio n (chemolysis alcohol)/n (urethane groups), in which n=molar amount) of 4.5 to 30, more preferably 5.0 to 20, most preferably 8.5 to 15. If the molar amount of the urethane groups is unknown (for instance because polyurethanes of unknown origin are to be reutilized), this can be determined easily by hydrolyzing a representative sample of the (poly)urethane provided in (A) and determining the amine value of the hydrolysis product. Since one mole of the amine is released per mole of cleaved urethane groups, the amine value can be used to ascertain the molar amount of urethane groups. The exact manner of performance of the hydrolysis is of course dependent on the nature of the (poly)urethane. With the aid of the information and literature references collated in [1], it is a simple matter for the person skilled in the art to find a suitable process regime.

The amine value indicates how many mg of potassium hydroxide are required to neutralize the free organic amines present in 1 g of substance. This covers primary, secondary and tertiary amino groups. The amino groups are weak bases. The solvent used is concentrated acetic acid (glacial acetic acid, 99% to 100%). The amine is protonated by the solvent and thus converted to the corresponding acid, which is now present as an ion pair with the deprotonated acid of the glacial acetic acid. The mixture is subsequently titrated with 0.1 molar perchloric acid as the titrant, in the course of which the perchloric acid displaces the anion of the solvent (glacial acetic acid). The perchloric acid consumed in the process is equated to the consumption of potassium hydroxide. The amine number is typically reported in milligrams of KOH per gram of analyzed sample and is calculated as follows:

$$\frac{AV}{mg ( KOH ) \cdot {g}^{- 1}} = \frac{\frac{V}{ml} \cdot [ {b}_{i} / ( mol \cdot {l}^{- 1} ) ] \cdot [ M ( KOH ) / ( g \cdot {mol}^{- 1} ) ] \cdot f}{\frac{m}{g}}$$

in which
AV represents the amine value,
V represents the volume of perchloric acid solution consumed,
m represents the mass of the titrated sample,
M (KOH) represents the molar mass of KOH (56.11 g. mol-1),
bi represents the molarity of the perchloric acid solution and
f represents the dimensionless factor (titer) of the perchloric acid solution.

According to the invention, the chemolysis alcohol is selected from (i) an aliphatic secondary or tertiary (preferably secondary) monoalcohol, (ii) an aliphatic primary monoalcohol bonded to two or three further carbon atoms at (at least) one carbon atom in the β position or γ position (preferably β position) to the hydroxy group of the monoalcohol, (iii) an aromatic monoalcohol or (iv) a mixture of two or more of the aforementioned chemolysis alcohols. The present invention further provides for the use of such a chemolysis alcohol in a chemolysis (chemical cleavage) of a urethane (especially polyurethane) based on an isocyanate component and an alcohol component to form a carbamate of an isocyanate of the isocyanate component and the chemolysis alcohol to reduce the formation of by-products in which (at least) one organic residue is bonded to a nitrogen atom via an aliphatic carbon atom (especially originating from the chemolysis alcohol or the alcohol component).

Suitable chemolysis alcohols are preferably iso-propanol, sec-butanol, iso-butanol, tert-butanol, 2-pentanol, 3-pentanol, iso-amyl alcohol (3-methyl-1-butanol), 2-methyl-2-butanol, neo-pentanol (2,2-dimethyl-1-propanol), 2-methyl-1-butanol, 3-methyl-2-butanol, 2-methyl-1-butanol, cyclopentanol, hexan-2-ol, hexan-3-ol, 2-methylpentan-1-ol, 2-methylpentan-2-ol, 2-methylpentan-3-ol, 4-methylpentan-2-ol, 3-methylpentan-1-ol, 3-methylpentan-2-ol, 3-methylpentan-3-ol, 2,2-dimethylbutan-1-ol, 3,3-dimethylbutan-1-ol, 3,3-dimethylbutan-2-ol, 2,3-dimethylbutan-1-ol, 2,3-dimethylbutan-2-ol, 2-ethylbutan-1-ol, 4-methylpentan-2-ol, cyclohexanol, phenol, 2-ethylhexan-1-ol or mixtures of two or more of the aforementioned chemolysis alcohols. Particular preference is given to iso-propanol, sec-butanol, iso-butanol, 2-pentanol, 3-pentanol, iso-amyl alcohol (3-methyl-1-butanol), neo-pentanol (2,2-dimethyl-1-propanol), 2-methyl-1-butanol, 3-methyl-2-butanol, pentaerythritol, 2-methyl-1-butanol, cyclopentanol, hexan-2-ol, hexan-3-ol, 2-methylpentan-1-ol, 2-methylpentan-3-ol, 4-methylpentan-2-ol, 3-methylpentan-1-ol, 3-methylpentan-2-ol, 2,2-dimethylbutan-1-ol, 3,3-dimethylbutan-1-ol, 3,3-dimethylbutan-2-ol, 2,3-dimethylbutan-1-ol, 2-ethylbutan-1-ol, 4-methylpentan-2-ol, cyclohexanol, phenol and 2-ethylhexanol.

Among the nonaromatic chemolysis alcohols, the use of which is generally preferred, preference is given to sec-butanol, iso-butanol, iso-amyl alcohol (3-methyl-1-butanol), neo-pentanol (2,2-dimethyl-1-propanol), 2-methyl-1-butanol, cyclopentanol, 4-methylpentan-2-ol, cyclohexanol and 2-ethylhexanol. Particular preference is given to iso-propanol, 2-butanol, neo-pentanol (2,2-dimethyl-1-propanol), 2-methyl-1-butanol, iso-amyl alcohol (3-methyl-1-butanol), 2-ethyl-1-butanol and cyclohexanol, very particular preference to iso-propanol, 2-butanol, neo-pentanol (2,2-dimethyl-1-propanol), 2-methyl-1-butanol, 2-ethyl-1-butanol and cyclohexanol.

The chemolysis may, but need not necessarily, be performed in the presence of a catalyst.

In one embodiment, the addition of a chemolysis catalyst is deliberately dispensed with. If a catalyst is used, it preferably comprises a carbonate, a hydrogencarbonate, a hydroxide, an orthophosphate, a monohydrogenorthophosphate, a metaphosphate, an orthovanadate (where all the aforementioned chemolysis catalysts are preferably used in the form of their sodium or potassium salts), a titanium alkoxide (especially tetra-n-butyl titanate, Ti(O-nBu)₄), a tertiary amine (especially 1,4-diazabicyclo(2.2.2) octane, “DABCO”), cesium fluoride, a stannate (especially dibutyltin dilaurate, “DBTL”, or monobutyltin oxide, n-BuSn(O)OH, “MBTO”) or a mixture of two or more of the aforementioned chemolysis catalysts.

### Workup of the Chemolysis Product

The chemolysis affords a chemolysis product comprising the carbamate. The procedure for workup of the chemolysis product preferably involves, in a step (C), separating the carbamate formed in (B) from the chemolysis product using an extraction with an organic solvent, optionally with addition of water, and/or a solid-liquid phase separation to obtain a liquid alcohol phase (especially a polyol phase) as well as the carbamate. Excess chemolysis alcohol is separated off before or after the separation, preferably before.

Separation by solid-liquid phase separation is restricted to those carbamates that precipitate out of the chemolysis product in solid form (after cooling if appropriate). Whether this is the case depends on the exact nature of the carbamate, i.e. in particular on the nature of the (poly)urethane to be cleaved. However, separation by extraction is employable in any case. Separation of alcohols (especially polyols) and carbamates that are present alongside one another in a mixture in the product of an alcoholysis by extractive processes is known in principle in the prior art. Reference is made, for example, to international patent applications WO 2020/260387 A1 (use of very nonpolar organic solvents such as, in particular, hydrocarbons) and WO 2022/063764 A1 (use of moderately polar organic solvents such as, in particular, halogenated hydrocarbons in conjunction with an aqueous wash liquid).

In the context of the process of the invention, it is possible to use either halogen-substituted (especially chlorinated) organic solvents, for example halogen-substituted aliphatic hydrocarbons, halogen-substituted alicyclic hydrocarbons, halogen-substituted aromatic hydrocarbons or mixtures of two or more of the aforementioned organic solvents, or nonhalogenated solvents, for example aliphatic hydrocarbons, alicyclic hydrocarbons, aromatic hydrocarbons or mixtures of two or more of the aforementioned organic solvents. The choice of solvent under some circumstances is directed in the individual case particularly by the nature of the (poly)urethane to be cleaved, and can be ascertained by the person skilled in the art if required by simple preliminary experiments.

In the case of separation of the carbamate using an extraction, the carbamate is obtained as a solution; depending on the nature of the (poly)urethane used and the type of organic solvent used, it is obtained dissolved in the extraction solvent or—as described in WO 2020/260387 A1-in excess chemolysis alcohol or—as described in WO 2022/063764 A1-in an aqueous phase which is obtained after addition of an aqueous wash liquid to the extraction. Depending on the type of intended further processing of the carbamate, it is possible to use this solution directly. But it is likewise easily possible to isolate the carbamate from this solution by evaporation of the solvent and/or crystallization.

The carbamate thus obtained can then, optionally after purification, be converted further in a step (D) to obtain a chemical product, where (D) comprises one of the following reactions:
(D.I) hydrolysis of the carbamate in the presence or absence of a hydrolysis catalyst to form an amine corresponding to the isocyanate of the isocyanate component;
(D.II) hydrogenolysis of the carbamate in the presence of a hydrogenolysis catalyst to form an amine corresponding to the isocyanate of the isocyanate component;
(D.III) cleaving of the carbamate in the presence or absence of a carbamate cleavage catalyst to the isocyanate of the isocyanate component and the chemolysis alcohol; or
(D.IV) reaction of the carbamate with a polyol in the presence or absence of a catalyst to give a further OH-terminated carbamate, especially—when the starting urethane is a polyurethane—to give an OH-terminated prepolymer.

The reactions of (D.I) to (D.IV), hydrolysis, hydrogenolysis, carbamate cleavage and transurethanization, are well known per se in the prior art and will therefore only be outlined briefly here.

The hydrolysis of (D.I) gives the amine corresponding to the isocyanate of the isocyanate component. In principle, such a hydrolysis can be conducted analogously to the direct hydrolysis of polyurethanes (in this regard see the literature cited further up, especially the review article [1]). The amines thus formed can then be sent to any of the applications known in the specialist field for such amines; one involves phosgenating them again to give the corresponding isocyanates. The isocyanate thus obtained can then be sent back to the production of a (poly)urethane. The hydrolysis can be assisted by use of a hydrolysis catalyst. The following are especially suitable for this purpose:
(I) an (organic or inorganic) Brønsted base selected from (i) a hydroxide (especially sodium hydroxide, tetramethylammonium hydroxide, potassium hydroxide or tetrabutylammonium hydroxide), (ii) a carbonate (especially an alkali metal carbonate, for example sodium or potassium carbonate), (iii) a hydrogencarbonate (especially an alkali metal hydrogencarbonate such as sodium or potassium hydrogencarbonate), (iv) an orthophosphate or metaphosphate, preferably orthophosphate (especially alkali metal phosphate or alkali metal hydrogenphosphate) or (v) a mixture of two or more of the aforementioned Brønsted bases.
   and/or
(II) a urethanase, especially one of the urethanases described in EP 3 587 570 A1.

The amine corresponding to the isocyanate of the isocyanate component may also be obtained by the hydrogenolysis of the carbamate in (D.II) with hydrogen. A process that proceeds directly from polyurethanes and is also employable for the present step (D.II) is described in Hydrogenative Depolymerization of Polyurethanes Catalyzed by Manganese Pincer Complex by Viktorija Zubar et al., published in ChemSusChem 2022, 15, e202101606 [2]. Reference is likewise made to the literature cited in [2]. The possible uses of the amine are the same as outlined for (D.I). The hydrogenolysis is preferably assisted by use of a catalyst. A suitable hydrogenolysis catalyst is especially a catalyst comprising
palladium (especially Pd/C, PdCl₂ or Pd(OAc)₂), copper, nickel (especially Raney nickel), manganese (especially Mn complexes having a tridentate chelate ligand that binds via P and N donor atoms, and CO and/or halogen ligands) or platinum (especially platinum (IV) oxide).

The workup variants of (D.I) and (D.II) thus afford the amine corresponding to the isocyanate of the isocyanate component. This amine can be sent to any of the end uses known in the specialist field; in particular, it can be phosgenated and used in the production of new (poly)urethane.

However, it is also conceivable to obtain the isocyanate of the isocyanate component directly from the carbamate by cleaving the carbamate into the isocyanate of the isocyanate component and the chemolysis alcohol either by purely thermal means or in the presence of a carbamate cleavage catalyst (carbamate cleavage of (D.III)). In the case of a catalytic procedure, a suitable carbamate cleavage catalyst is especially:
(I) a metal-free or metal-containing Brønsted- or Lewis-acidic catalyst
   or
(II) a metal-free or metal-containing Brønsted- or Lewis-basic catalyst.

For further details, reference is made to W. Leitner et al., Carbon2Polymer-Chemical Utilization of CO₂ in the Production of Isocyanates, Chapter 4, “Carbamate Cleavage”, published in Chem. Ing. Tech. 2018, 90, 1504-1512, and the literature cited therein.

The workup variant of (D.III) directly affords the isocyanate of the isocyanate component, and so there is no need for another phosgenation. This isocyanate can be sent to any of the uses known in the specialist field; it can especially be reacted again with H-functional compounds to give polyaddition products, preferably (poly)urethanes. The carbamate cleavage of (D.III) may be the variant of choice especially when the isocyanate component of the urethane or polyurethane comprises and especially consists of tolylene diisocyanate (TDI) (i.e. does not comprise any further isocyanates aside from TDI).

The reaction of the carbamate from step (C) with a polyol to give a further OH-terminated carbamate of (D.IV) is a further means of further processing the carbamate. In chemical terms, this is a transurethanization and hence in principle the same kind of reaction as that which also forms the basis of the glycolysis of polyurethanes (in this regard, see the literature cited further up, especially the review article [1]). It is advantageously employable especially when the urethane provided in step (A) is a polyurethane. For this purpose, the carbamate is reacted with a polyol, with use of the OH groups of the polyol in a stoichiometric or superstoichiometric, especially slightly superstoichiometric, amount (e.g. a 5% to 10% excess on a molar basis) relative to the carbamate functionalities present. If the starting urethane is a polyurethane, which is preferred, this reaction will lead to OH-terminated prepolymers. These can be sent to any of the end uses known in the specialist field; they may especially be used as prepolymers for flexible foam and rigid foam applications, thermoplastic polyurethanes, coatings and adhesives.

The polyol used for step (D.IV) preferably has a boiling point above that of the chemolysis alcohol used. In a particularly preferred embodiment, the chemolysis alcohol is continuously removed from the reaction mixture by distillation during the reaction with the polyol. The reaction can optionally be conducted in the presence of a catalyst. The latter preferably comprises a carbonate, a hydrogencarbonate, a hydroxide, an orthophosphate, a metaphosphate, an orthovanadate (where all the aforementioned catalysts are preferably used in the form of their sodium or potassium salts), a titanium alkoxide (especially tetra-n-butyl titanate, Ti(O-nBu)₄), a tertiary amine (especially 1,4-diazabicyclo(2.2.2) octane, “DABCO”), cesium fluoride, a stannate (especially dibutyltin dilaurate, “DBTL”, or monobutyltin oxide, n-Bu—Sn(O)OH, “MBTO”) or a mixture of two or more of the aforementioned catalysts.

Typical suitable polyols are dihydric polyols (especially ethylene glycol, diethylene glycol, propylene glycol, dipropylene glycol, propane-1,3-diol, butane-1,4-diol, butene-1,4-diol, butyne-1,4-diol, neopentyl glycol, pentane-1,5-diol, methylpentanediols (for example 3-methylpentane-1,5-diol), hexane-1,6-diol, octane-1,8-diol, decane-1,10-diol, dodecane-1,12-diol, bis(hydroxymethyl)cyclohexanes (for example 1,4-bis(hydroxymethyl)cyclohexane), triethylene glycol, tetraethylene glycol, polyethylene glycols, dipropylene glycol, tripropylene glycol, polypropylene glycols, dibutylene glycol and polybutylene glycols), trihydric polyols (especially trimethylolpropane, glycerol, trishydroxyethyl isocyanurate), tetrahydric polyols (especially pentaerythritol) and polyols that can be obtained from renewable raw materials (especially sorbitol, hexitol, sucrose, starch, starch hydrolyzates, cellulose, cellulose hydrolyzates and hydroxy-functionalized fats and oils, especially castor oil), and all modification products of these aforementioned polyols with different amounts of ε-caprolactone. Polyols used for step (D.IV) may likewise be polyether polyols, especially those having a number-average molar mass Mn determined to DIN55672-1 (2016-03) in the range from 18 g/mol to 8000 g/mol and a functionality (calculated from the H-functional starters used in the production of the polyether polyols) of 2 to 3. Preference is given to polyether polyols formed from repeat ethylene oxide and propylene oxide units, preferably having a proportion of 35% to 100% propylene oxide units, more preferably having a proportion of 50% to 100% propylene oxide units. These may be random copolymers, gradient copolymers, alternating copolymers or block copolymers of ethylene oxide and propylene oxide.

In all four variants, the chemolysis alcohol used is released again and can be recovered by distillation and used again in the chemolysis.

The more selectively the carbamate is formed in step (B), the more advantageous it is to implement the above-described variants for further processing of the carbamate. This is especially true of variant (D.III), in which the isocyanate is obtained without the amine intermediate. But even in the other variants, it may be advantageous when the formation of the carbamate proceeds as selectively as possible because this in many cases facilitates the subsequent workup, especially the separation of the constituents of the chemolysis product from one another. One selectivity-reducing reaction is therefore in particular the direct (“uncontrolled”) formation of the amine in step (B). In order to suppress the formation of the amine, it is therefore preferable to use chemolysis alcohols with a minimum water content. In particular, it is preferable that the chemolysis alcohol used, based on its total mass, contains not more than 0.500% by mass, preferably not more than 0.200% by mass, more preferably not more than 0.050% by mass, most preferably not more than 0.005% by mass, of water, which can be achieved by drying measures known per se if required. The mass of the chemolysis alcohol for the purposes of the present invention is considered in each case to mean the total mass including any water present. The water content of the chemolysis alcohol is determinable if required by Karl Fischer titration; this is the crucial method for the purposes of the present invention. Karl Fischer titration has been described many times and is well known to the person skilled in the art. Various possible configurations of the basic principle of Karl Fischer titration generally give results with sufficiently good agreement within the scope for the purposes of the present invention. In the case of doubt, Karl Fischer titration as described in DIN 51 777, Part 1, March 1983, is crucial for the purposes of the present invention.

If the recovery of the carbamate is unimportant in respect of the desired (poly)urethane recycling, it is likewise possible to hydrolyze the chemolysis product formed in step (B) directly, i.e. without separating off the carbamate formed. In this step, step (B) comprises a step (B.I), hydrolysis of the carbamate formed in (B) in the presence or absence of a hydrolysis catalyst to form an amine by reaction of the (unchanged) chemolysis product obtained in (B) with water to obtain a hydrolyzed product mixture. The workup in this case comprises a step (C.I), extraction of the hydrolyzed product mixture with an organic (especially halogenated) solvent to obtain an amine phase and a liquid alcohol phase (especially a polyol phase). Suitable organic (especially halogenated) solvents are the same as described above in connection with the extraction of the carbamate.

In the context of the process of the invention, there are thus in principle two possible routes for the workup of the chemolysis product, namely a first with obtaining of the carbamate, followed by separate further processing thereof and of the resultant liquid alcohol phase, and a second with direct hydrolysis of the chemolysis product. With regard to the workup of the chemolysis product, the process of the invention therefore comprises either
(α) a step (C), separation of the carbamate formed in (B) from the chemolysis product using an extraction with an organic solvent, optionally with addition of water, and/or a solid-liquid phase separation to obtain a liquid alcohol phase (especially a polyol phase) as well as the carbamate, where the carbamate separated off in (C), optionally after purification, is preferably converted further in a step (D) to obtain a chemical product, where (D) comprises one of the following reactions:
   (D.I) hydrolysis of the carbamate in the presence or absence of a hydrolysis catalyst to form an amine corresponding to the isocyanate of the isocyanate component;
   (D.II) hydrogenolysis of the carbamate in the presence of a hydrogenolysis catalyst to form an amine corresponding to the isocyanate of the isocyanate component;
   (D.III) cleaving of the carbamate in the presence or absence of a carbamate cleavage catalyst to the isocyanate of the isocyanate component and the chemolysis alcohol; or
   (D.IV) reacting the carbamate with a polyol in the presence or absence of a catalyst to give a further OH-terminated carbamate;
   where variants (D.I), (D.II) and (D.IV) are preferred and variant (D.I) is particularly preferred;
      or
(β) a step (B.I), hydrolysis of the carbamate formed in (B) in the presence or absence of a hydrolysis catalyst to form an amine by reaction of the chemolysis product obtained in (B) with water to obtain a hydrolyzed product mixture, and a step (C.I), extraction of the hydrolyzed product mixture with an organic (especially halogenated) solvent to obtain an amine phase and a liquid alcohol phase (especially a polyol phase).

With regard to the liquid alcohol phase from (C) or (C.I), it is preferable to distill and/or strip the latter in a step (E) to obtain (at least) one chemical product selected from (i) an alcohol of the alcohol component and/or (ii) a reaction product formed from an alcohol of the alcohol component in the chemolysis (B). The alcohols and/or reaction products thus obtained can be sent to any of the end uses known in the specialist field of such compounds. In particular, it is possible to use recovered polyether polyols in the production of new polyurethanes and reaction products of polyester polyols in the production of new polyester polyols.

The invention described above is elucidated in detail hereinafter by examples.

### EXAMPLES

### Chemicals

| Name | Sourced from | Purity |
|---|---|---|
| phenyl isocyanate | Sigma-Aldrich | >98% |
| MeOH | Sigma-Aldrich | >99.9% |
| iPrOH | Sigma-Aldrich | 99.5% |
| EG | Sigma-Aldrich | 99.8% |
| 2-ethoxyethanol | Alfa Aesar | 99% |
| aniline | Riedel-de Haën | 99.5% |
| THF | Sigma-Aldrich (Merck) | n.a. |
| tetradecane | Sigma-Aldrich (Merck) | >99.0% |
| Na₂CO₃ | Sigma-Aldrich | >99.5% |
| 2-butanol | Fluka | >99.5% |
| 2,2′-dimethyl-1- | Sigma-Aldrich | 99% |
| propanol | | |
| 2-methyl-1-butanol | Sigma-Aldrich | ≥99% |
| 3-methyl-1-butanol | Sigma-Aldrich | ≥99% |
| 2-ethyl-1-butanol | Sigma-Aldrich | 98% |
| cyclohexanol | Acros Organics | 98% |
| Ethylene glycol | Sigma-Aldrich | 99.8% |
| diethylene glycol | Sigma-Aldrich | ≥99% |

### Analysis

### GC Method

Gas chromatography with a flame ionization detector (GC-FID) was used for quantitative evaluation of the samples. An Agilent 8890 GC system was used with an SSL inlet (275° C., split ratio 80:1 with a constant flow rate of 5 ml/min), an HP-5 GC column (30 m, internal diameter 320 μm, film thickness 0.25 μm) and H₂ as carrier gas. At the start of the measurement, the temperature is kept constant at 60° C. for 0.5 min and then increased to 300° C. with a ramp of 20° C./min. On attainment of that temperature, a temperature of 300° C. is maintained for a further 10 min. The solvent used was tetrahydrofuran (THF).

The following formula was used for calculation of the GC yields and conversion by means of an internal tetradecane standard:

$${m}_{i} = \frac{{m}_{int std} * {A}_{i} * kf}{{A}_{int std}}$$

mᵢ=mass of substance i
M_{int std}=mass of internal standard
Aᵢ=area integral of substance i
A_{int std}=area integral of internal standard
kf=correction factor (FID response factor)

In this way, the proportions by mass of 2-ethoxyethyl-N-phenylurethane (substrate; see the section below), the target carbamate and aniline were quantified. A kf of 1 was assumed for the N-alkylated aniline derivatives.

### EXPERIMENTAL

The model reaction used was the reaction of 2-ethoxyethyl-N-phenylurethane,

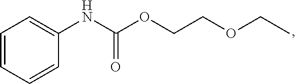

(substrate henceforth) with various chemolysis alcohols to form corresponding transurethanization products (target carbamates henceforth).

### Synthesis of 2-ethoxyethyl N-phenylcarbamate

To an initial charge of 2-ethoxyethanol (59.8 g, 64.3 mL, 0.66 mol, 6.00 equiv.) in a 250 ml round-bottom flask was rapidly added phenyl isocyanate (13.2 g, 12.1 mL, 0.11 mol, 1.00 equiv.) while stirring (at 500 rpm) and room temperature. The reaction solution was then stirred at 100° C. for 4.5 h. Once the reaction solution had cooled down to room temperature, CHCl₃ (~60 mL) was added and extracted with demineralized water (3×50 mL). The organic phase was washed with saturated saline solution and dried over MgSO₄. The volatile components were removed under reduced pressure, before 2-ethoxyethyl N-phenylcarbamate was dried under high vacuum over 72 h and isolated as a yellowish, viscous liquid (16.7 g, 72.2%).

### Alcoholysis Reactions in High-Pressure Autoclaves

A glass insert equipped with a magnetic stirrer is filled with catalyst (optional (see tables 1 and 2), 28 g, 1.0% by mass based on the total mass of substrate and chemolysis alcohol), substrate (700 mg), chemolysis alcohol (2.1 g) and the internal tetradecane standard (20 mg), and placed into the stainless steel autoclave (capacity 20 mL). This is purged three times with N₂ (establishment of a nitrogen pressure of 50 bar, followed by decompression to ambient pressure) and closed, before the pressure is increased by adding nitrogen to 10 bar. The autoclave is heated in a preheated aluminum cone while stirring (900 rpm) at 200° C. for 4 h. Subsequently, the autoclave is cooled in an ice bath for 10 min and then decompressed. The reaction mixture is diluted with THF (2 mL), filtered through a syringe filter (Chromafil O-20/15 MS) and analyzed by GC-FID.

### Examples 1 to 36

Table 1 below collates the results of the alcoholysis reactions.

TABLE 1
Alcoholysis of a glycol-based urethane as substrate with different chemolysis
alcohols at 200° C. and a reaction time of 120 to 360 min^{[a]}

| | | | | | | | | N- |
|---|---|---|---|---|---|---|---|---|
| | Chemolysis | b.p. | | t | Y_{substrate} | Y_{TC} | Yₐₙᵢₗᵢₙₑ | alkylation |
| Ex. | alcohol | [° C.] | Cat. | [min] | [%]^{[b]} | [%]^{[b]} | [%]^{[b]} | [%]^{[c]} |
| 1 | MeOH | 65 | — | 240 | 0.98 | 96.2 | 2.7 | 0.18 |
| (C^{[d]}) | | | | | | | | |
| 2 | MeOH | 65 | Na₂CO₃ | 240 | 0.00 | 84.6 | 14.7 | 0.66 |
| (C) | | | | | | | | |
| 3 | EtOH | 78 | — | 240 | 2.14 | 88.9 | 8.9 | 0.10 |
| (C) | | | | | | | | |
| 4 | EtOH | 78 | Na₂CO₃ | 240 | 2.76 | 81.8 | 15.2 | 0.29 |
| (C) | | | | | | | | |
| 5 | n-PrOH | 97 | — | 240 | 4.91 | 94.3 | 0.8 | 0.01 |
| (C) | | | | | | | | |
| 6 | n-PrOH | 97 | Na₂CO₃ | 240 | 4.88 | 87.9 | 7.2 | 0.03 |
| (C) | | | | | | | | |
| 7 | iso-PrOH | 82 | — | 240 | 8.54 | 89.2 | 2.3 | 0.00 |
| 8 | iso-PrOH | 82 | Na₂CO₃ | 240 | 7.89 | 84.8 | 7.3 | 0.00 |
| 9 | 2-butanol | 99 | — | 120 | 61.39 | 36.6 | 2.0 | 0.00 |
| 10 | 2-butanol | 99 | Na₂CO₃ | 120 | 10.44 | 78.7 | 10.8 | 0.00 |
| 11 | 2,2-dimethyl- | about 113 | — | 120 | 58.65 | 39.4 | 1.9 | 0.00 |
| | 1-propanol | | | | | | | |
| 12 | 2,2-dimethyl- | about 113 | Na₂CO₃ | 120 | 8.33 | 82.4 | 9.3 | 0.00 |
| | 1-propanol | | | | | | | |
| 13 | 2-methyl- | 129 | — | 120 | 35.85 | 63.2 | 0.9 | 0.00 |
| | 1-butanol | | | | | | | |
| 14 | 2-methyl- | 129 | Na₂CO₃ | 120 | 4.62 | 89.6 | 5.7 | 0.00 |
| | 1-butanol | | | | | | | |
| 15 | 3-methyl- | 131 | — | 120 | 41.58 | 57.4 | 1.0 | 0.00 |
| | 1-butanol | | | | | | | |
| 16 | 3-methyl- | 131 | Na₂CO₃ | 120 | 3.73 | 90.4 | 5.9 | 0.00 |
| | 1-butanol | | | | | | | |
| 17 | 2-ethyl- | 149 | — | 120 | 54.63 | 44.4 | 0.9 | 0.00 |
| | 1-butanol | | | | | | | |
| 18 | 2-ethyl- | 149 | Na₂CO₃ | 120 | 4.44 | 90.7 | 4.9 | 0.00 |
| | 1-butanol | | | | | | | |
| 19 | cyclo- | 161 | — | 360 | 15.64 | 80.0 | 4.3 | 0.02 |
| | hexanol | | | | | | | |
| 20 | cyclo- | 161 | Na₂CO₃ | 360 | 7.50 | 81.6 | 10.9 | 0.05 |
| | hexanol | | | | | | | |
| 21 | Ethylene | 197 | — | 120 | 23.81 | 4.8 | 38.1 | 33.3 |
| (C) | glycol | | | | | | | |
| 22 | diethylene | 244 | — | 120 | 0.00 | 0.0 | 29.0 | 71.0 |
| (C) | glycol | | | | | | | |

TABLE 2
Alcoholysis of a glycol-based urethane as substrate with different chemolysis
alcohols at 200-220° C. and a reaction time of 240-480 min (higher
temperatures and longer reaction times compared to table 1)^{[a]}

| | | | | | | | | | N- |
|---|---|---|---|---|---|---|---|---|---|
| | Chemolysis | b.p. | | T | t | Y_{substrate} | Y_{TC} | Yₐₙᵢₗᵢₙₑ | alkylation |
| Ex. | alcohol | [° C.] | Cat. | [° C.] | [min] | [%]^{[b]} | [%]^{[b]} | [%]^{[b]} | [%]^{[c]} |
| 23 | MeOH | 65 | — | 200 | 480 | 2.09 | 94.8 | 1.56 | 1.59 |
| (C^{[d]}) | | | | | | | | | |
| 24 | MeOH | 65 | Na₂CO₃ | 200 | 480 | 0.82 | 69.9 | 15.4 | 13.9 |
| (C) | | | | | | | | | |
| 25 | MeOH | 65 | — | 220 | 240 | 4.49 | 82.1 | 10.8 | 2.63 |
| (C) | | | | | | | | | |
| 26 | MeOH | 65 | Na₂CO₃ | 220 | 240 | 0.97 | 34.2 | 48.0 | 16.8 |
| (C) | | | | | | | | | |
| 27 | n-PrOH | 97 | Na₂CO₃ | 200 | 480 | 4.53 | 79.1 | 13.4 | 3.01 |
| (C) | | | | | | | | | |
| 28 | n-PrOH | 97 | Na₂CO₃ | 220 | 240 | 2.88 | 81.8 | 14.0 | 1.42 |
| (C) | | | | | | | | | |
| 29 | iso- | 82 | — | 200 | 480 | 8.08 | 78.9 | 13.0 | 0.00 |
| | PrOH | | | | | | | | |
| 30 | iso- | 82 | Na₂CO₃ | 200 | 480 | 18.3 | 78.6 | 3.05 | 0.00 |
| | PrOH | | | | | | | | |
| 31 | iso- | 82 | — | 220 | 240 | 9.46 | 86.2 | 4.38 | 0.00 |
| | PrOH | | | | | | | | |
| 32 | iso- | 82 | Na₂CO₃ | 220 | 240 | 8.53 | 77.4 | 14.1 | 0.00 |
| | PrOH | | | | | | | | |
| 35 | sec- | 99 | Na₂CO₃ | 200 | 480 | 8.73 | 76.5 | 14.8 | 0.00 |
| | butanol | | | | | | | | |
| 36 | sec- | 99 | Na₂CO₃ | 220 | 240 | 8.75 | 72.48 | 18.77 | 0.00 |
| | butanol | | | | | | | | |

### Elucidations for the Table:

[a] Substrate: PhNH—CO—O—(CH₂)₂—OEt; mass ratio of chemolysis alcohol to substrate 3:1. If a catalyst is used, it is used in a proportion by mass of 1.0%, based on the total mass of substrate and chemolysis alcohol. The reaction conditions are optimized to maximized yield of the target carbamate in the case of the uncatalyzed reaction with methanol (ex. 1).
[b] Percentage of the substrate/target carbamate/aniline based on molar amount in the product in relation to the amount of substrate used:

$$\begin{matrix}{Y}_{substrate} = ( 100 \% \cdot [ {n ( substrate )}_{product} / {n ( sunstrate )}_{used} ] or \\ {Y}_{TC} = 100 \% \cdot [ {n ( target carbamate )}_{product} / {n ( substrate )}_{used} ] or \\ {Y}_{aniline} = 100 \% \cdot [ n ( {aniline}_{product} / {n ( substrate )}_{used} ] .\end{matrix}$$

[c] Quotient of
   (i) the sum total of the area percentages (GC) of the detected N-alkylated aniline derivatives R¹R²NC₆H⁵ (where R¹ is an alkyl moiety of the chemolysis alcohol or a (CH₂)₂—OEt group from the substrate and R² is either hydrogen, an alkyl moiety of the chemolysis alcohol or a (CH₂)₂—OEt group from the substrate, where R¹ may also be the same as R²) and
   (ii) the sum total of the area percentages (GC) of the detected N-alkylated aniline derivatives, of free aniline, of the substrate and of the target carbamate;
multiplied by 100.
[d] C=comparative example.

As shown by the examples from tables 1 and 2, the use of the simple linear C₁ to C₄ alcohols leads to formation of N-alkylated by-products (examples 1 to 6). Replacement of n-propanol with iso-propanol leads to reduction of the level of N-alkylated by-products to zero (examples 7 to 8). Even in the case of longer reaction times and higher reaction temperatures, with or without the use of an additional catalyst, no N-alkylation is observed when the alcohols of the invention are used (table 2).

The use of methyl- and ethyl-substituted butanols—in the presence of a catalyst—in spite of non-optimized reaction conditions and even half the reaction time compared to ex. 2, leads to very good yields of target carbamate in the product mixture of around 90%, likewise without N-alkylation (examples 14, 16 and 18). With iso-propanol, a comparable yield is achieved even without catalyst (ex. 7).

Glycols, which are traditionally used as chemolysis alcohols, give enormous amounts of N-alkylated by-products and are unusable under the reaction conditions chosen (examples 21 and 22).

1. A process for chemolyzing a urethane based on an isocyanate component and an alcohol component by reaction with a chemolysis alcohol to form a carbamate of an isocyanate of the isocyanate component and of the chemolysis alcohol, comprising the steps of:
(A) providing the urethane and
(B) chemolyzing the urethane from (A) with the chemolysis alcohol in the presence or absence of a chemolysis catalyst, where the chemolysis alcohol at 1013 mbar has a boiling point of not more than 200° C., and where a mass ratio of the chemolysis alcohol to the urethane, m(chemolysis alcohol)/m(urethane), is in the range from 0.50 to 15, to form a chemolysis product comprising the carbamate,
wherein the chemolysis alcohol is selected from (i) an aliphatic secondary or tertiary monoalcohol, (ii) an aliphatic primary monoalcohol bonded to two or three further carbon atoms, at at least one carbon atom in the β position or γ position to the hydroxy group of the monoalcohol, (iii) an aromatic monoalcohol or (iv) a mixture of two or more of the aforementioned chemolysis alcohols.

2. The process of claim 1, comprising (C) separation of the carbamate formed in (B) from the chemolysis product using an extraction with an organic solvent and/or a solid-liquid phase separation to obtain a liquid alcohol phase as well as the carbamate.

3. The process of claim 2, in which the carbamate separated off in (C), optionally after purification, is converted further in a step (D) to obtain a chemical product, where (D) comprises one of the following reactions:
(D.I) hydrolysis of the carbamate in the presence or absence of a hydrolysis catalyst to form an amine corresponding to the isocyanate of the isocyanate component;
(D.II) hydrogenolysis of the carbamate in the presence of a hydrogenolysis catalyst to form an amine corresponding to the isocyanate of the isocyanate component;
(D.III) cleaving of the carbamate in the presence or absence of a carbamate cleavage catalyst to the isocyanate of the isocyanate component and the chemolysis alcohol; or
(D.IV) reaction of the carbamate with a polyol in the presence or absence of a catalyst to produce a further OH-terminated carbamate.

4. The process of claim 1, further comprising the steps of:
(B.I) hydrolyzing the carbamate formed in (B) in the presence or absence of a hydrolysis catalyst to form an amine by reaction of the chemolysis product obtained in (B) with water to obtain a hydrolyzed product mixture; and
(C.I) extracting the hydrolyzed product mixture with an organic solvent to obtain an amine phase and a liquid alcohol phase.

5. The process of claim 2, wherein the liquid alcohol phase from (C) or (C.I), in a step (E), is distilled and/or stripped to obtain a chemical product selected from (i) an alcohol of the alcohol component and/or (ii) a reaction product formed from an alcohol of the alcohol component in the chemolysis (B).

6. The process of claim 1, wherein the chemolysis of the urethane is performed in the absence of a chemolysis catalyst.

7. The process of claim 1, wherein the chemolysis of the urethane is performed in the presence of a chemolysis catalyst, where the chemolysis catalyst comprises a carbonate, a hydrogencarbonate, a hydroxide, an orthophosphate, a monohydrogenorthophosphate, a metaphosphate, an orthovanadate, a titanium alkoxide, a tertiary amine, cesium fluoride, a stannate or a mixture of two or more of the aforementioned chemolysis catalysts.

8. The process of claim 1, wherein the chemolysis alcohol is selected from iso-propanol, sec-butanol, iso-butanol, tert-butanol, 2-pentanol, 3-pentanol, iso-amyl alcohol (3-methyl-1-butanol), 2-methyl-2-butanol, neo-pentanol (2,2-dimethyl-1-propanol), 2-methyl-1-butanol, 3-methyl-2-butanol, 2-methyl-1-butanol, cyclopentanol, hexan-2-ol, hexan-3-ol, 2-methylpentan-1-ol, 2-methylpentan-2-ol, 2-methylpentan-3-ol, 4-methylpentan-2-ol, 3-methylpentan-1-ol, 3-methylpentan-2-ol, 3-methylpentan-3-ol, 2,2-dimethylbutan-1-ol, 3,3-dimethylbutan-1-ol, 3,3-dimethylbutan-2-ol, 2,3-dimethylbutan-1-ol, 2,3-dimethylbutan-2-ol, 2-ethylbutan-1-ol, 4-methylpentan-2-ol, cyclohexanol, phenol, 2-ethylhexan-1-ol or a mixture of two or more of the aforementioned chemolysis alcohols.

9. The process claim 1, wherein the chemolysis alcohol is selected from (i) an aliphatic secondary or tertiary monoalcohol, (ii) an aliphatic primary monoalcohol bonded to two or three further carbon atoms at a carbon atom in the β position or γ position to the hydroxy group of the monoalcohol, or (iv) a mixture of two or more of the aforementioned chemolysis alcohols.

10. The process of claim 9, wherein the chemolysis alcohol is selected from sec-butanol, iso-butanol, iso-amyl alcohol (3-methyl-1-butanol), neo-pentanol (2,2-dimethyl-1-propanol), 2-methyl-1-butanol, cyclopentanol, 4-methylpentan-2-ol, cyclohexanol and 2-ethylhexanol.

11. The process of claim 1, wherein the isocyanate component comprises an isocyanate selected from:—
phenyl isocyanate, tolylene diisocyanate, the di- and polyisocyanates of the diphenylmethane series, pentane 1,5-diisocyanate, hexamethylene 1,6-diisocyanate, isophorone diisocyanate, diisocyanatodicyclohexylmethane, xylylene diisocyanate, para-phenylene diisocyanate or a mixture of two or more of the aforementioned isocyanates.

12. The process of claim 1, wherein the alcohol component comprises a mono- and/or polyol selected from:
a polyether monool, a polyether polyol, a polyester polyol, a polyetherester polyol, a polyacrylate polyol, a polycarbonate polyol, a polyethercarbonate polyol or a mixture of two or more of the aforementioned polyols.

13. The process of claim 1, wherein step (B) is performed within a temperature range from 150° C. to 270° C.

14. The process of claim 1, wherein m(chemolysis alcohol)/m(urethane) in (B) is adjusted to 0.50 to 4.5.

15. (canceled)

16. The process of claim 1, wherein m(chemolysis alcohol)/m(urethane) in (B) is adjusted to 1.0 to 4.5.
